(19) United States
(12) Patent Application Publication
(10) Pub. No.: US 2021/0251622 A1
(43) Pub. Date: Aug. 19, 2021

(54) ANCHORED SUTURE STAPLE AND METHOD FOR IMPLANTING

(71) Applicant: David S. Ryan, Athens, GA (US)

(72) Inventor: David S. Ryan, Athens, GA (US)

(21) Appl. No.: 17/000,358

(22) Filed: Aug. 23, 2020

Related U.S. Application Data
(60) Provisional application No. 62/890,784, filed on Aug. 23, 2019.
(60) Provisional application No. 62/904,456, filed on Sep. 23, 2019.

(51) Int. Cl. A61B 17/04; A61B 17/064; A61F 2/08

(52) U.S. Cl. CPC A61B 17/0401; A61B 17/064; A61F 2/0811; A61B 2017/0647; A61F 2002/0817; A61B 2017/0641; A61B 2017/0412

(57) ABSTRACT

A staple-suture combination that includes a plurality of anchors, wherein each of the anchors are constructed from suture material and a cinching stitch such that the cinching stitch can be pulled thereby causing the suture material to cinch up and secure the anchor below the cortex of a bone and further including a mattress stitch that extends over a surface of a section of tissue thereby securing the tissue to the bone. The cinching stitch and/or the mattress stitch including barbs on an outer surface prevent the cinching stitch and/or mattress stitch from loosening.

### BACKGROUND

Life is good when the skeletal system and the muscular system work together. It is when the muscular system and the skeletal system separate at one or more critical points that problems arise. Several areas of the body are more susceptible to this category of injury, such as the rotator cuff and the knee. The rotator cuff is a group of muscles and tendons that form a cuff over the shoulder joint. These muscles and tendons hold the arm in its joint and help the shoulder joint to move. Rotator cuff tears may occur as a result of a single injury or may occur due to progressive degeneration or wear, such as is common from repetitive overhead activity or heavy lifting over a prolonged period of time.

A group of four muscles and their respective tendons present in the shoulder region which act in harmony to stabilize the shoulder are known as rotator cuff muscles. Any injury leading to a tear in the muscle or the tendon calls for an immediate treatment. Reattaching the fibers of the muscle and tendons in cases of severe tear by opening up the shoulder joint is known as rotator cuff repair surgery. As illustrated in FIG. 1A, the four muscles constituting the rotator cuff group are:

Supraspinatus muscle

Infraspinatus muscle

Teres minor muscle

Subscapularis muscle

These muscles originate from the scapula (shoulder bone) and are inserted on the humerus (upper arm bone), forming a cuff at the shoulder joint. These muscles bring about rotation of the upper arm and stabilize the shoulder joint.

There are over 600,000 rotator cuff surgeries being performed in the US each year, and the number is rising at approximately 5% each year. Not all of these surgeries are successful: the success rate for surgery is dependent on many factors, the two most important of which are the size of the original tear and the age of the patient. Estimates have put the re-tear (or non-healing) rate at an average of 50%.

The lack of apparent success for this type of shoulder surgery presents an enormous opportunity if the success rate can be improved. The cost of readmission, revision surgery and further physical therapy can often amount to multiple (tens of) thousands of dollars: a reduction on the probability of surgical failure is therefore an extremely attractive proposition to patients, surgery centers and payers.

FIG. 1B illustrates a normal rotator cuff. FIG. 1C illustrates a tear in the supraspinatus tendon. FIG. 1D illustrates a typical surgical solution to repair the tear of the supraspinatus tendon.

The human knee exists at a joint between the femur and tibia. This joint probably receives the most rigorous treatment of all the joints in the body. Typically, the stress on the knee joint can be due to the pressures applied when walking, running, jumping or performing most athletic activities, as well as from external impact common in athletic activity, falls, accidents, etc. Fortunately, the knee joint includes a cushion that helps to absorb some of this stress. This cushion is referred to as the meniscus.

The meniscus is a c-shaped soft cartilage tissue that exists between the femur bone and tibia bone. There are two menisci in each knee, the medial meniscus and the lateral meniscus. The medial meniscus is located on the inner side of the knee and the lateral meniscus is located on the outer side of the knee. FIG. 1 is a drawing showing the location of the medial and lateral menisci. The meniscus is held in place by being connected or attached to the surface of the tibia. The ends of the c-shaped tissue of the meniscus are referred to as the roots. The roots of the meniscus are attached to the tibia surface, or the articular cartilage that covers the tibia. FIG. 2 is a drawing illustrating a healthy meniscus that is properly attached to the tibia. The menisci operate to cushion or absorb the stress or pressure that occurs between the femur bone and the tibia, as well as the articular cartilage that covers the ends of these bones within the knee joint. As such, while the menisci are attached to the tibia, the femur rests on the upper surface of the menisci. It can thus be appreciated that the menisci operate as a shock absorber to minimize the stress and create a pocket to hold the femur in position relative to the tibia. The protection provided by the meniscus helps to alleviate onset of arthritis and ablation of the femur and tibia from frictional contact.

The pocket created by menisci operates to provide stability in the knee joint. In essence, the meniscus can be viewed as a wedge that operates to maintain the position of the femur and tibia relative to each other. If the meniscus is worn away or diminished, the knee joint can become unstable. Thus, it should be understood and appreciated that the meniscus is a very important element of human anatomy, and if it is injured or damaged, it should be immediately repaired.

There are two common types of meniscus injuries. The first is when a tear occurs in the meniscus. This is referred to as a meniscus tear. The second is similar to the first, but it is a tear that fully separates (avulses) or partially separates the root of the meniscus from where it is connected to the articular cartilage or bone. This is referred to as a meniscus root tear.

A meniscus tear can take many forms, some that are repairable and some that are not. For meniscus tears, the reparation procedure typically involves suturing across the tear to prevent further tearing and to promote healing of the tear.

A meniscus root tear is a bit more complicated. The attachment of the meniscal roots to the tibia are critical in maintaining the normal shock absorbing function of the meniscus and the support for the femur. If a meniscal tear or a meniscal root tear is left untreated, meniscal extrusion can occur rendering the meniscus nonfunctional and resulting in degenerative arthritis. Also, the meniscus is not self-regenerative and thus, any damage to the meniscus should be treated immediately to prevent the damage from resulting in further tissue loss and other ill side effects.

For meniscal root tears, where the root of the meniscus avulses or partially avulses from the surface of the tibia, sutures have been used to connect to or through the meniscus and then the meniscus is secured in place by transtibial fixation of the sutures. FIG. 2 is a drawing illustrating the use of transtibial fixation. It can thus be understood that a procedure to correct a meniscus root tear requires a posterior arthroscopy portal to be opened and then the drilling of a tunnel through the tibia. Finally, the sutures that are attached to the meniscus, pulled through the tunnel and attached to an anchor, stop or button on the posterior side of the tibia. This technique is very difficult and time consuming. What is needed in the art is a technique to repair meniscus root injuries that reduces the complexity and time required to perform the procedure.

Further, what is needed in the art is a technique to repair other muscle root injuries or other injuries where the muscular system separates from the skeletal system.

### BRIEF SUMMARY

The present disclosure is directed towards a device that enables soft tissue and skeletal repairs to be performed more efficiently and reliably. As a non-limiting embodiment, the present disclosure is directed towards a technique to enable muscle or tendon tears to be repaired more efficiently and without the requirement of a posterior arthroscopy portal or using direct or indirect fixation through a tunnel. Advantageously, the embodiments presented within this disclosure reduce the complexity of the tear repair (such as supraspinatus tendon or meniscus root tear repair) and reduce the potential of further damage resulting from drilling a tunnel for transtibial indirect fixation (i.e., transtibial fixation for meniscus root repair). It will be understood, that while the present disclosure focuses on rotator cuff and meniscus repairs, the techniques, devices, methods and features presented herein can also be applied to any of a variety of other soft tissue repair, including muscles, tendons, ligaments, etc. that have become fully or partially separated, or avulsed, from the bone.

One embodiment includes a staple-suture combination that can be used to attach to the muscle or tendon and to the bone (i.e., humerus for rotator cuff repair or tibia for meniscus root tear). In one embodiment, a two-pronged staple is utilized with a suture extending between the prongs of the staple. In another embodiment, a three-pronged staple is used with sutures extending between each of the prongs. In yet another embodiment, a single-pronged staple can be used with a pad or head constructed of a soft material but having a surface space substantially large enough to hold the muscle or tendon in place. In one or more of these embodiments, the suture may be a tube with a barbed suture passing there-through that can be embedded into the bone and then by pulling the barbed suture, the suture tube is gathered to create the anchor. These and other embodiments are described further in the detailed description section.

### BRIEF DESCRIPTION OF THE SEVERAL VIEWS OF THE DRAWING

FIG. 1A is a drawing showing the muscles and tendons involved in the rotator cuff.

FIG. 1B is a drawing of a normal shoulder.

FIG. 1C is a drawing of a rotator cuff injury in which the supraspinatus tendon is torn.

FIG. 1D is a drawing of a typical repair of a torn supraspinatus tendon.

FIG. 2A is a drawing showing the location of the medial and lateral menisci.

FIG. 2B is a drawing illustrating the meniscus attached to the tibia.

FIG. 3 is a conceptual diagram illustrating a two-pronged embodiment of the meniscus root repair device.

FIG. 4 is a conceptual diagram illustrating a two-pronged staple in the implanted state.

FIG. 5 is a conceptual diagram illustrating a three-pronged embodiment of the meniscus root repair staple.

FIG. 6 is a conceptual diagram illustrating a three-pronged staple in the implanted state.

FIG. 7 is a conceptual diagram illustrating a single-pronged embodiment of the meniscus root repair staple.

FIG. 8 is a conceptual diagram illustrating a single-pronged staple in the implanted state.

FIG. 9 is a conceptual diagram illustrating another embodiment of a three-pronged meniscus root repair staple.

FIG. 10 is a conceptual diagram illustrating the three-pronged staple of FIG. 9 in the implanted state.

FIG. 11A illustrates a round prong 1110A with a tapered end 1144A.

FIG. 11B illustrates a square or rectangular prong 1110B with a tapered end 1144B.

FIG. 11C illustrates a triangular prong 1110C with a tapered end 1144C.

FIG. 12 is an exemplary embodiment of a tool that can be used to implant a meniscus root repair staple.

FIG. 13 is another exemplary embodiment of a tool that can be used to implant a meniscus root repair staple.

FIG. 14 is a conceptual diagram illustrating a two-pronged embodiment of the meniscus root repair device that includes one or more additional free sutures that are attached to the staple device.

FIG. 15 is a prior art depiction of an anchor.

FIG. 16 is a prior art depiction of a deployed anchor.

FIG. 17 illustrates another embodiment of a suture staple using multiple anchors as depicted in FIG. 15.

FIG. 18 illustrates the suture staple of FIG. 17 in a deployed state.

FIG. 19 illustrates an awl or punch to prepare for placement of a staple suture.

FIG. 20 illustrates an implant tool for deploying the suture staple of FIG. 17.

FIG. 21 illustrates a cannula that can be used in conjunction with the implant tool of FIG. 17 and the awl of FIG. 19.

FIG. 22 illustrates a cross-sectional view of the cannula of FIG. 19 taken at line 22-22.

FIG. 23 illustrates another embodiment of the two-anchor suture staple.

FIG. 24 illustrates the suture staple embodiment of FIG. 23 in a deployed configuration.

FIG. 25 is yet another embodiment of a two-anchor suture staple.

FIG. 26 illustrates the suture staple of FIG. 25 in a deployed or implanted state.

FIG. 27 illustrates the application of the embodiment of the suture staple of FIGS. 17-18 in use for a double rotator cuff repair.

FIG. 28 illustrates the application of the embodiment of the suture staple of FIGS. 25-26 in use for a double rotator cuff repair.

FIG. 29 illustrates an embodiment of a suture stable of a two-anchor suture staple with a barbed suture.

FIG. 30 illustrates the suture staple of FIG. 29 in a deployed or implemented state.

FIG. 31 is yet another embodiment of a two-anchor suture staple.

FIG. 32 illustrates the suture staple of FIG. 31 in a deployed state.

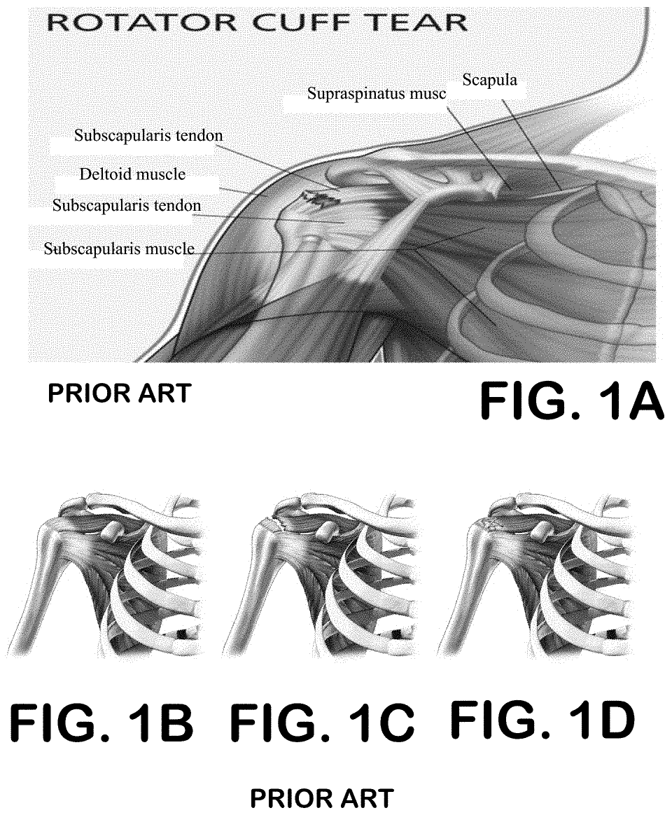

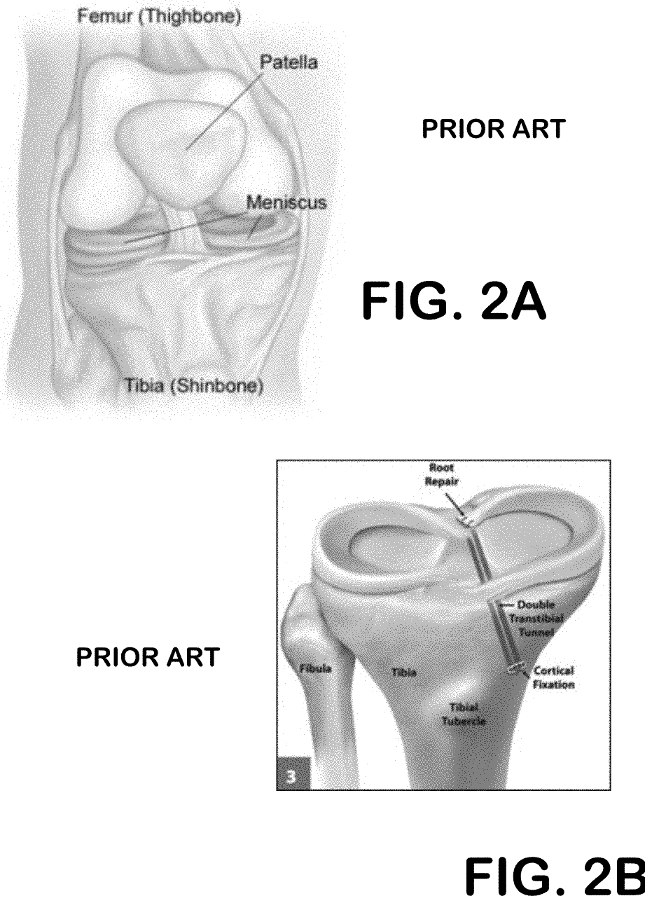

### DETAILED DESCRIPTION OF EMBODIMENTS

The present invention, as well as features and aspects thereof, is directed towards providing a suture device, a suturing technique and deployment devices for providing soft tissue and skeletal repairs, and more specifically, a sutured staple that secures an avulsed or partially avulsed tissue to the bone without having to be attached or indirectly fixed in a tunneling manner. While the various embodiments, features and aspects of the inventions presented herein focus on meniscus and rotator cuff repair, it should be appreciated that these examples are provided by way of illustration to aid in understanding of the embodiments, features and aspects and should not be viewed as limiting examples.

In the description and claims of the present application, each of the verbs, “comprise”, “include” and “have”, and conjugates thereof, are used to indicate that the object or objects of the verb are not necessarily a complete listing of members, components, elements or parts of the subject or subjects of the verb.

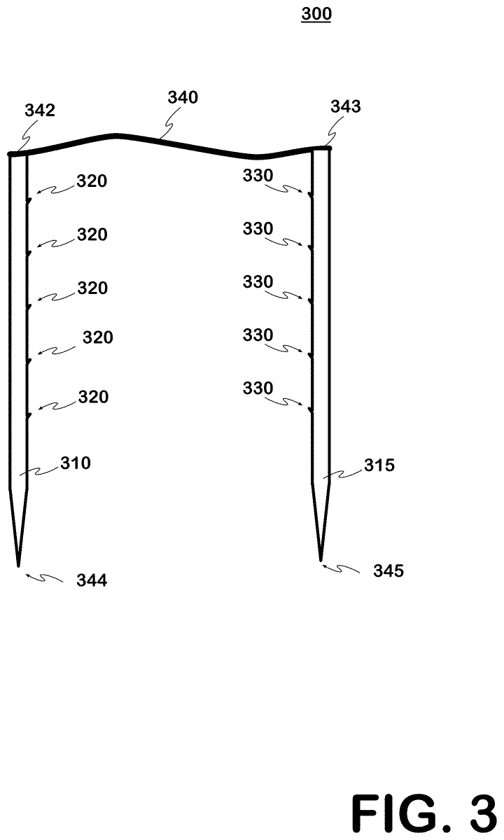

FIG. 3 is a conceptual diagram illustrating a two-pronged embodiment of the tissue repair device. In the illustrated embodiment, a staple-like device 300 is illustrated as including two prongs 310 and 315. On one end, the prongs 310 and 315 are tapered (344 and 345 respectively) to facilitate penetration of the prongs 310 and 315 into the bone. Along at least one tangential surface (for round cross-sectional prongs) or at least one surface or side of the prongs 310 and 315 a plurality of protrusions or barbs 320 and 330 respectively may extend from the surface of the prongs 310 and 330. The barbs 320 and 330 are angled such that they minimize the impedance of driving the prongs 310 and 315 into the bone but greatly inhibit the ability of the prongs to exit the bone once driven therein. It should be appreciated that in this embodiment and other embodiments or equivalents thereof, the terms “bone” and “tissue” are used to describe the operation. However, it should be appreciated that the term “bone” may be used to describe any element that is suitable for receiving the prongs or anchor and the term “tissue” may include, without limitation, muscles, tendons, ligaments, dermis or any other tissue related material that is being anchored.

On the upper point of the prongs 310 and 315 a suture 340 is attached at points 342 and 343 respectively. The suture is a soft material, such as a thread, nylon or other suitable material such as is common in the art with sutures (i.e., most modern sutures are synthetic, including the absorbable polyglycolic acid, polylactic acid, Monocryl and polydioxanone as well as the non-absorbable nylon, polyester, PVDF and polypropylene as non-limiting examples), whereas the prongs 310 and 315 can be manufactured from a rigid material, such as stainless steel, composite or cobalt steel as non-limiting examples.

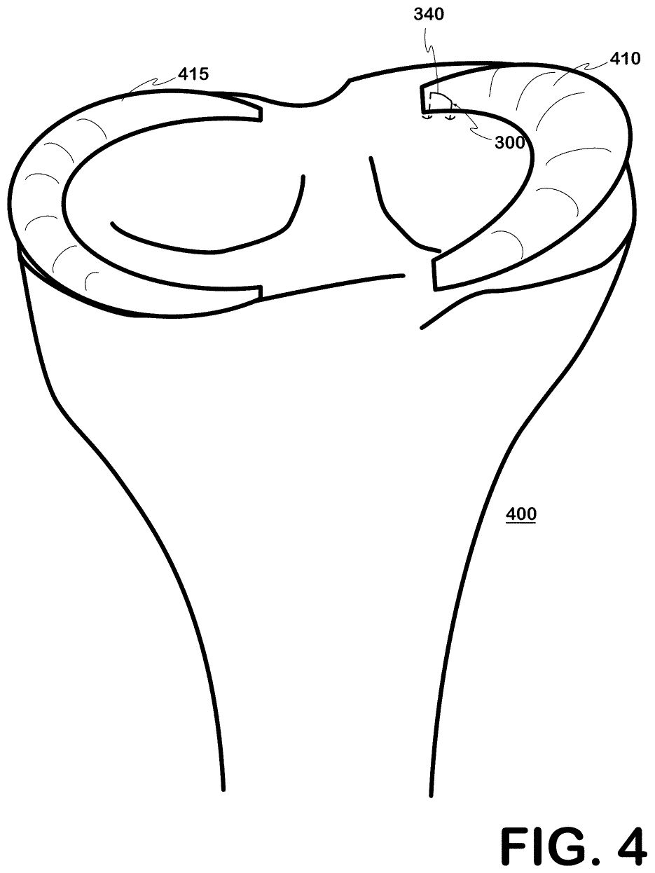

FIG. 4 is a conceptual diagram illustrating a two-pronged staple in the implanted state utilized in a meniscus repair as a non-limiting example. It can be seen that the tibia bone 400 includes a medial meniscus 410 and a lateral meniscus 415. The two-pronged staple 300 is illustrated as piercing the root of the medial meniscus and penetrating into the tibia bone. In operation, the two-pronged staple 300 is implanted into a subject by using a tool to position the staple 300 at the desired position, and then through the tool, applying force against the prongs 310 and 315 to drive the prongs through the surface of the tibia and into the tibia. Thus, once implanted, the suture 340 holds the meniscus against the tibia and the prongs hold the meniscus in place relative to the tibia.

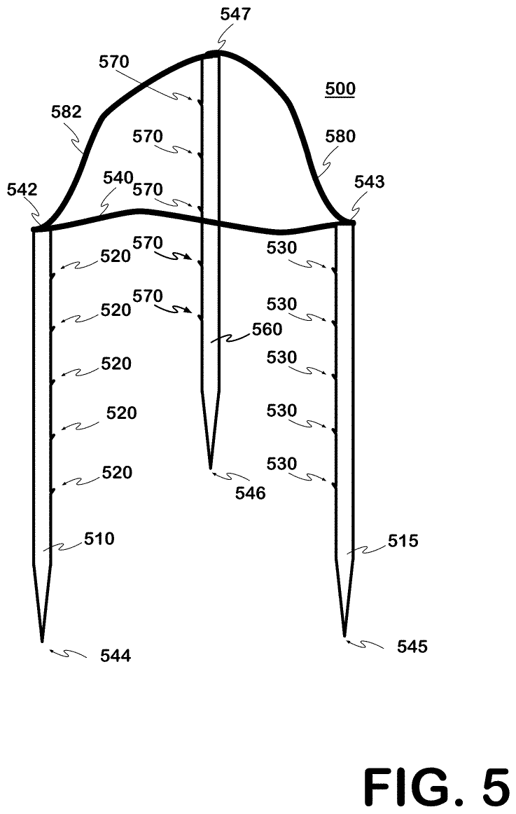

FIG. 5 is a conceptual diagram illustrating a three-pronged embodiment of a tissue repair staple. In the illustrated embodiment, a staple-like device 500 is illustrated as including three prongs 510, 515 and 560. On one end, the prongs 510, 515 and 560 are tapered (544, 545 and 546 respectively) to facilitate penetration of the prongs 510, 515 and 560 through the tissue and into the bone. Along at least one tangential surface (for round cross-sectional prongs) or at least one surface or side of the prongs 510, 515 and 560 a plurality of protrusions or barbs 520, 530 and 570 respectively may extend from the surface of the prong 510, 515 and 560. The barbs 520, 530 and 570 are angled such that they minimize the impedance of driving the prongs 510, 515 and 560 into the bone but greatly inhibit the ability of the prongs to exit the tibia once driven therein.

On the upper point of the prongs 510, 515 and 560, three sutures are attached with suture 340 being attached at points 542 and 543, suture 580 being attached at points 543 and 547 and suture 582 being attached at points 542 and 547. The suture is a soft material, such as a thread, nylon or other suitable material such as is common in the art with sutures, whereas the prongs 510, 515 and 560 can be manufactured from a rigid material, such as stainless steel, composite or cobalt steel as non-limiting examples.

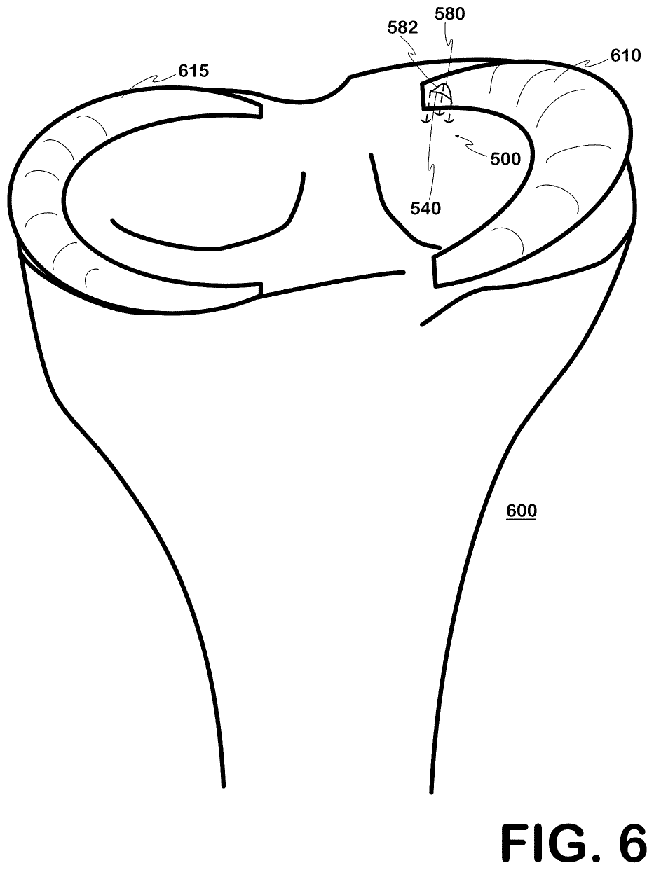

FIG. 6 is a conceptual diagram illustrating a three-pronged staple in the implanted state for a meniscus repair as a non-limiting example. It can be seen that tibia 600 includes a medial meniscus 610 and a lateral meniscus 615. The three-pronged staple 500 is illustrated as piercing the root of the medial meniscus 610 and penetrating into the tibia bone 600. In operation, the three-pronged staple 500 is implanted into a subject by using a tool to position the staple 500 at the desired position, and then through the tool, applying force against the prongs 510, 515 and 560 to drive the prongs through the surface of the tibia and into the tibia 600. Thus, once implanted, the sutures 540, 580 and 582 hold the meniscus against the tibia and the prongs 510, 515 and 560 hold the meniscus in place relative to the tibia 600.

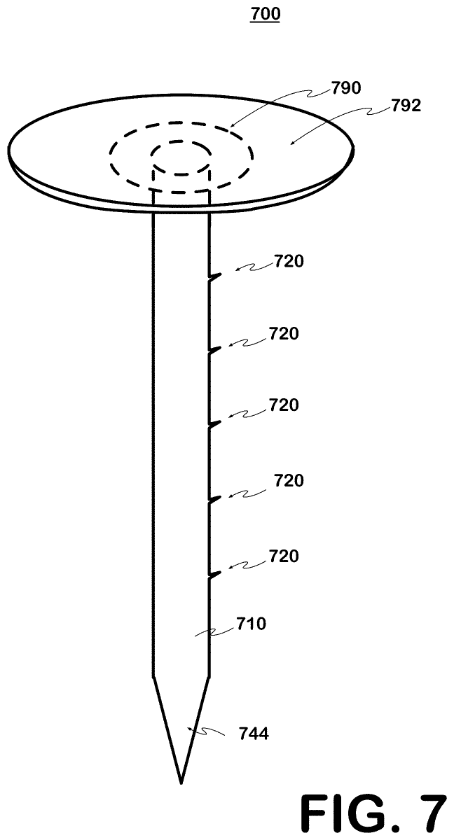

FIG. 7 is a conceptual diagram illustrating a single-pronged embodiment of a tissue repair staple. In the illustrated embodiment, a staple-like device 700 is illustrated as including a single prong 710. On one end, the prong 710 is tapered (744) to facilitate penetration of the prong 710 through the meniscus and into the tibia. Along at least one tangential surface (for round cross-sectional prongs), or at least one surface or side of the prong 710, a plurality of protrusions or barbs 720 may extend from the surface of the prong 710. The barbs 720 are angled such that they minimize the impedance of driving the prong 710 into the tibia but greatly inhibit the ability of the prong to exit the tibia once driven therein.

On the upper point of the prong 710 is a flat, nail-head-like structure 790. The nail-head 790 may be encased in a soft material 792 that operates to provide a soft material on the upper side that any neighboring bone can rest against and a soft material on the bottom side that rests against the tissue and holds it in place. The prong 710 may be manufactured with a rigid material, such as stainless steel, composite or cobalt steel as non-limiting examples. The encasement can be manufactured from a cloth, a mesh, silicone or other suitable material as a non-limiting example.

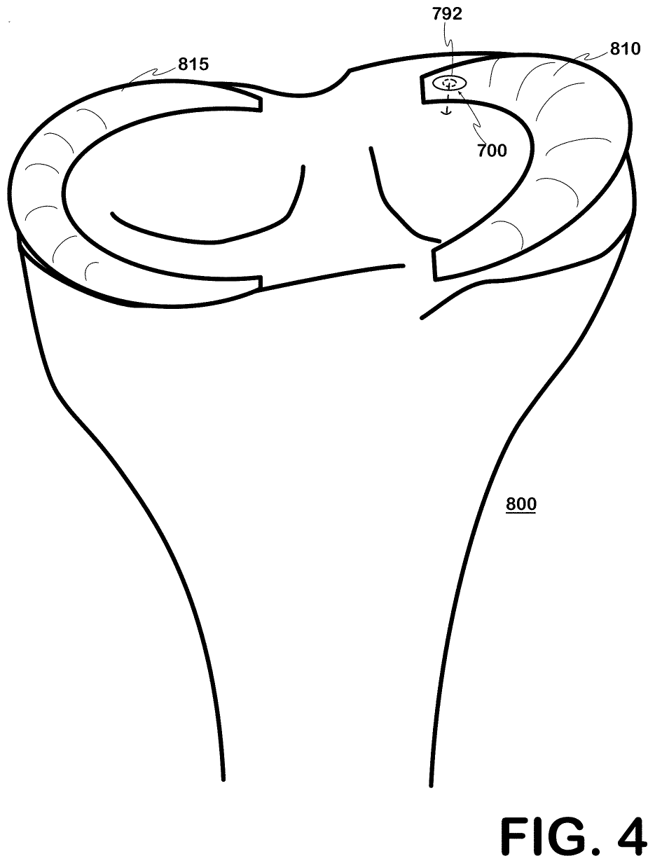

FIG. 8 is a conceptual diagram illustrating the single-pronged staple in the implanted state for a meniscus repair as a non-limiting example. It can be seen that tibia 800 includes a medial meniscus 810 and a lateral meniscus 815. The single-pronged staple 700 is illustrated as piercing the root of the medial meniscus 810 and penetrating into the tibia bone 800. In operation, the single-pronged staple 700 is implanted into a subject by using a tool to position the staple 700 at the desired position, and then through the tool, applying force against the prong 700 to drive the prong through the surface of the tibia and into the tibia 600. Thus, once implanted, the encasement 792 operates like a suture and holds the meniscus against the tibia 800, while the prong 700 holds the meniscus in place relative to the tibia 800.

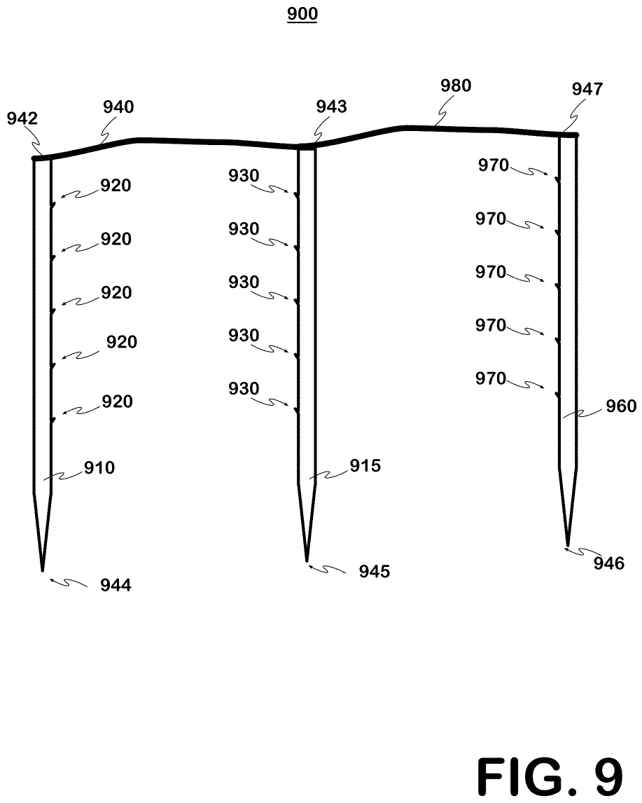

FIG. 9 is a conceptual diagram illustrating another embodiment of a three-pronged tissue repair staple. In the illustrated embodiment, a staple-like device 900 is illustrated as including three prongs 910, 915 and 960. On one end, the prongs 910, 915 and 960 are tapered (944, 945 and 946 respectively) to facilitate penetration of the prongs 910, 915 and 960 through the tissue and into the bone. Along at least one tangential surface (for round cross-sectional prongs), or at least one surface or side of the prongs 910, 915 and 960, a plurality of protrusions or barbs 920, 930 and 970 respectively may extend from the surface of the prong 910, 915 and 960. The barbs 920, 930 and 970 are angled such that they minimize the impedance of driving the prongs 910, 915 and 960 into the bone but greatly inhibit the ability of the prongs to exit the bone once driven therein.

On the upper point of the prongs 910, 915 and 960, two sutures are attached with suture 940 being attached at points 942 and 943 and suture 980 being attached at points 943 and 947. The suture is a soft material, such as a thread, nylon or other suitable material such as is common in the art with sutures, whereas the prongs 910, 915 and 960 can be manufactured from a rigid material, such as stainless steel, composite or cobalt steel as non-limiting examples.

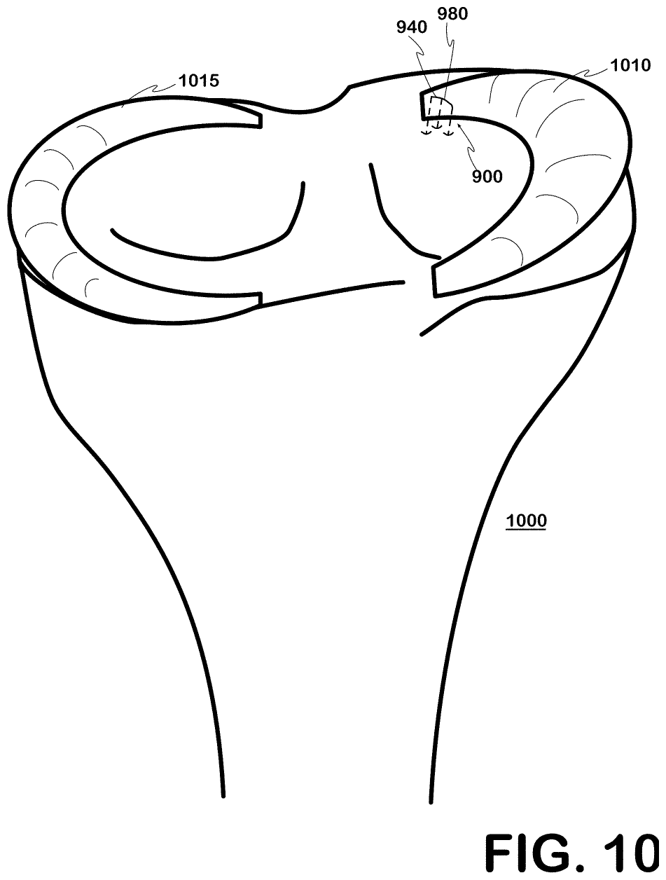

FIG. 10 is a conceptual diagram illustrating the three-pronged staple of FIG. 9 in the implanted state for a meniscus repair as a non-limiting example. It can be seen that tibia 1000 includes a medial meniscus 1010 and a lateral meniscus 1015. The three-pronged staple 1000 is illustrated as piercing the root of the medial meniscus 1010 and penetrating into the tibia bone 1000. In operation, the three-pronged staple 1000 is implanted into a subject by using a tool to position the staple 1000 at the desire position, and then through the tool, applying force against the prongs 910, 915 and 960 to drive the prongs through the surface of the tibia and into the tibia 1000. Thus, once implanted, the sutures 940 and 980 hold the meniscus against the tibia and the prongs 910, 915 and 960 hold the meniscus in place relative to the tibia 1000.

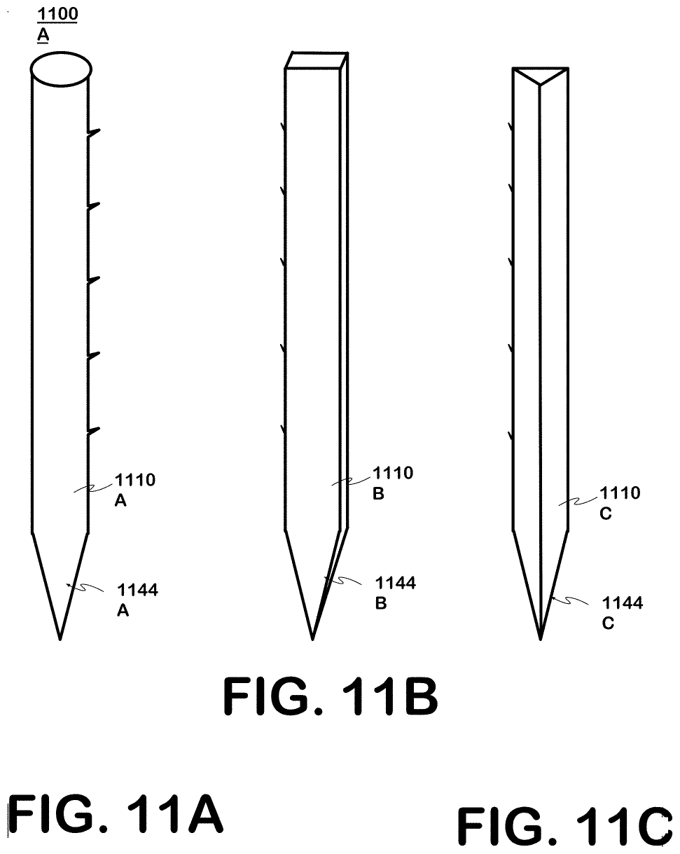

FIG. 11A, FIG. 11B and FIG. 11C illustrate three exemplary shapes that the prongs in any of the disclosed embodiments and equivalents thereof may take, as non-limiting examples. FIG. 11A illustrates a round prong 1110A with a tapered end 1144A. FIG. 11B illustrates a square cross-section or rectangular cross-section prong 1110B with a tapered end 1144B. FIG. 11C illustrates a triangular cross-section shaped prong 1110C with a tapered end 1144C. It should be appreciated that any one of the illustrated prong shapes as well as other prong shapes can be utilized in any of the embodiments presented herein and equivalents thereof. Further, it should be appreciated that while the illustrated embodiments present the use of 5 barbs on one side of the prongs, any number of barbs can be utilized and the barbs may be present on one or more sides of the prong.

In the various embodiments, the suture attached to the ends of the prongs can be attached with an adhesive, a sonic weld, tied in an indentation of the prong, or threaded through a hole in the top of the prong as a few non-limiting examples. In operation, the prongs are driven in the bone to a depth that allows the end of the prongs to be slightly below the top surface of the tissue, thus allowing the suture to be located above the tissue while the top of the prong is isolated from neighboring bones. The staples implanted in this manner will prevent the rigid surface of the prong from ablating any adjacent bones due to friction.

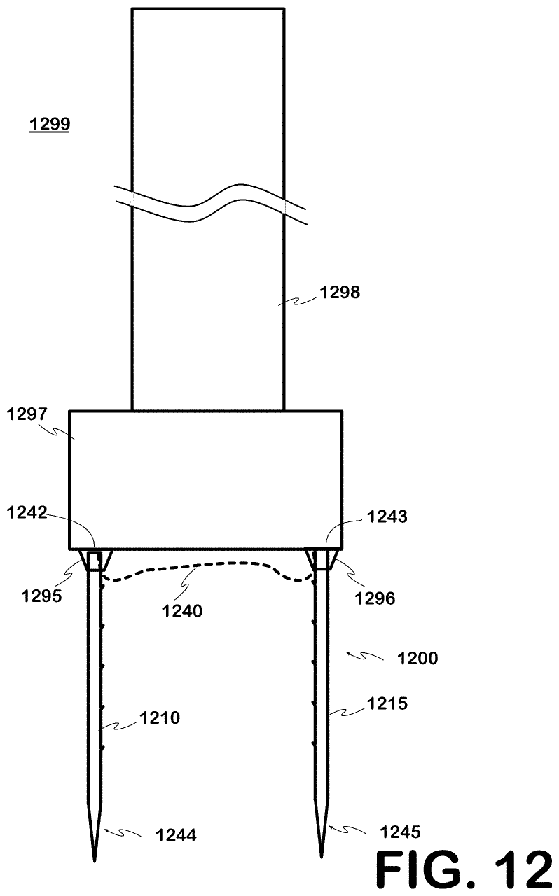

FIG. 12 is an exemplary embodiment of a tool that can be used to implant a tissue repair staple. The tool 1299 in the illustrated embodiment is configured for implanting a two-pronged staple 1200 but, it should be appreciated that the same techniques can be applied to a staple with any number of prongs. The illustrated two-pronged staple includes prongs 1210 and 1215, each being tapered respectively at ends 1244 and 1245.

The upper end 1242 of the prong 1210 and the upper end 1243 of prong 1215 are inserted into prong holders 1295 and 1296 respectively. The suture 1240 extends out of the bottom of the holders 1295 and 1296 and between the prongs 1210 and 1215. The prong holders 1295 and 1296 are attached to a bottom surface of base 1297. A shaft 1298 is attached to an upper surface of the base 1297 and extends upwardly from the surface of the base 1297. In operation, an incision is made over the area where the staple is to be implanted. The staple 1200 is then inserted into the prong holders 1295 and 1296. The tool 1299 is then used to insert the staple bearing end through the incision and position the staple over the tissue and bone at the desired location. A blunt force instrument is then used to drive the prongs 1210 and 1215 of the staple 1200 through the tissue and into the bone. The prongs are driven such that the holder 1295 and 1296 extend fully below the upper surface of the tissue. Thus, when the tool 1299 is extracted, the staple 1200 remains in position with the upper ends of the prongs existing below the surface of the tissue and the suture 1240 extending up through the tissue and across the upper surface between the prongs.

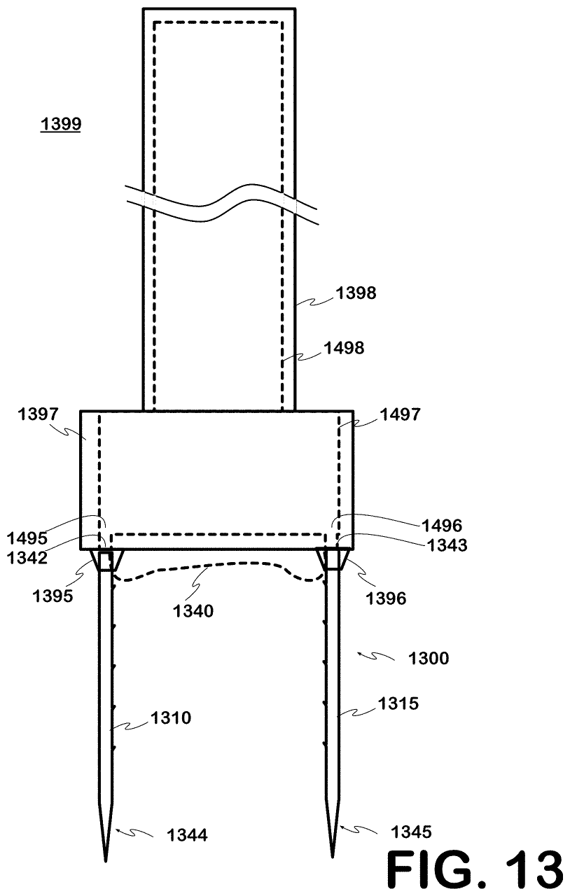

FIG. 13 is another exemplary embodiment of a tool that can be used to implant a tissue repair staple. The tool 1399 in the illustrated embodiment is configured for implanting a two-pronged staple 1300 but, it should be appreciated that the same techniques can be applied to a staple with any number of prongs. The illustrated two-pronged staple 1300 includes prongs 1310 and 1315, each being tapered respectively at ends 1344 and 1345.

Similar to the embodiment illustrated in FIG. 12, the upper end 1342 of the prong 1310 and the upper end 1343 of prong 1315 are inserted into prong holders 1395 and 1396 respectively. The prong holders 1395 and 1396 are attached to a bottom surface of base 1397. A hollow shaft 1398 is attached to an upper surface of the base 1397 and extends upwardly from the surface of the base 1397. In operation, an incision is made over the area where the staple is to be implanted. The staple 1300 is then inserted into the prong holders 1395 and 1396. The tool 1399 is then used to insert the staple bearing end through the incision and position the staple over the tissue at the desired location. A blunt force instrument is then used to drive the prongs 1310 and 1315 of the staple 1300 through the tissue and into the bone. The prongs are driven such that the holder 1395 and 1396 extend fully or partially below the upper surface of the meniscus. At this point the hollow shaft 1398 can be lifted thus revealing an inner shaft 1498. The inner shaft 1498 is affixed to block 1497 which is moveably positioned within a hollow area of base 1397. The bottom of block 1497 includes two protrusions 1495 and 1496 that align with holes in the bottom surface of the base 1397 right above the ends 1342 and 1343 of prongs 1310 and 1315 respectively. Using a blunt force instrument, the inner shaft 1498 can be struck, thus driving the protrusions or pins 1495 and 1496 through the prong holders 1395 and 1396 to further drive the prongs below the surface of the tissue. Thus, when the tool 1399 is extracted, the staple 1300 remains in position with the upper ends of the prongs existing below the surface of the tissue and the suture 1340 extending up through the tissue and across the upper surface between the prongs.

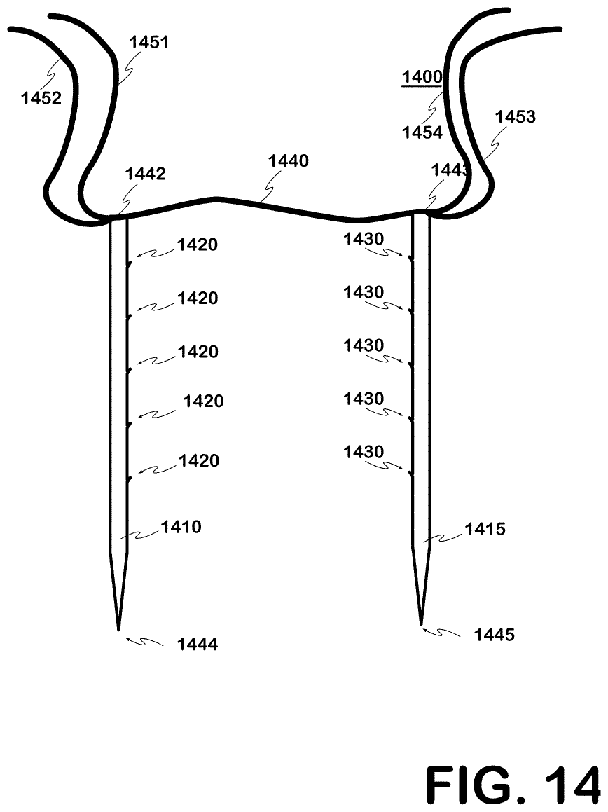

FIG. 14 is a conceptual diagram illustrating a two-pronged embodiment of the tissue repair device that includes one or more additional free sutures that are attached to the staple device. In the illustrated embodiment, a staple-like device 1400 is illustrated as including two prongs 1410 and 1415. On one end, the prongs 1410 and 1415 are tapered (1444 and 1445 respectively) to facilitate penetration of the prongs 1410 and 1415 into the bone. Along at least one tangential surface (for round cross-sectional prongs) or at least one surface or side of the prongs 1410 and 1415 a plurality of protrusions or barbs 1420 and 1430 respectively extend from the surface of the prong 1410 and 1430. The barbs 1420 and 1430 are angled such that they minimize the impedance of driving the prongs 1410 and 1415 into the bone but greatly inhibit the ability of the prongs to exit the bone once driven therein.

On the upper point of the prongs 1410 and 1415 a suture 1440 is attached at points 1442 and 1443 respectively. The suture is a soft material, such as a thread, nylon or other suitable material such as is common in the art with sutures (i.e., most modern sutures are synthetic, including the absorbable polyglycolic acid, polylactic acid, Monocryl and polydioxanone as well as the non-absorbable nylon, polyester, PVDF and polypropylene as non-limiting examples), whereas the prongs 1410 and 1415 can be manufactured from a rigid material, such as stainless steel, a composite material or cobalt steel as non-limiting examples. In addition to suture 1440, one or more free sutures may also be attached to the staple device (as depicted in FIG. 14). In operation, when the staple device is implanted into the bone, not only will there be a horizontal mattress type suture 1440 between the prongs, but there will be one or more additional suture(s) 1451, 1452, 1453 and 1454, as a non-limiting example, that can also be utilized. A notable application for this addition would be in double row rotator cuff repair, where the suture staple is the medial row fixation and a free suture attached to the staple is then brought over the rotator cuff in a crisscross fashion and secured laterally via standard techniques as the lateral row fixation as is common for “suture bridge double row repair” or “double row rotator cuff repair”.

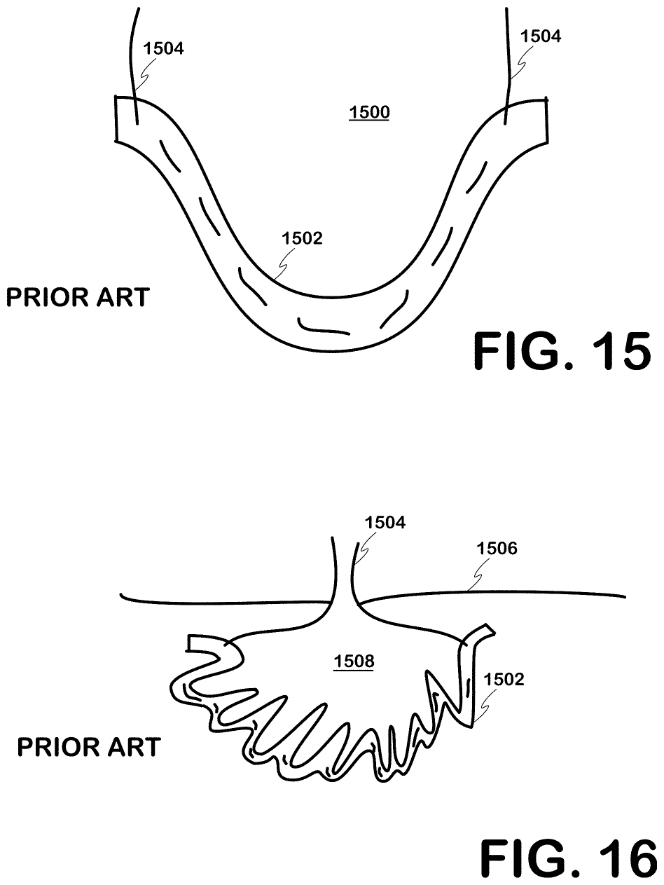

FIG. 15 is a prior art depiction of an anchor. In practice, an anchor is utilized to penetrate the cortex, or the cortical bone, and create a device that can be used to secure other elements to the bone. The cortical bone is the dense outer surface of bone that forms a protective layer around the internal cavity. This type of bone, also known as compact bone, makes up nearly 80% of skeletal mass and is imperative to body structure and weight bearing because of its high resistance to bending and torsion. The anchor 1500 includes a hollow tube 1502 and a cinch stitch 1504. In operation, the anchor 1500 is driven through the cortex 1506 and into the internal cavity 1508 such that the entire tube 1502 is below the cortex.

FIG. 16 is a prior art depiction of a deployment of the anchor illustrated in FIG. 15. Once the anchor 1500 is driven through the cortex 1506, the cinch stitch 1504 is pulled thereby causing the tube 1502 to ball up. As a result, the balled-up tube 1502 is larger than the opening through the cortex 1506 and thus, the anchor 1500 is securely held in place within the internal cavity 1508.

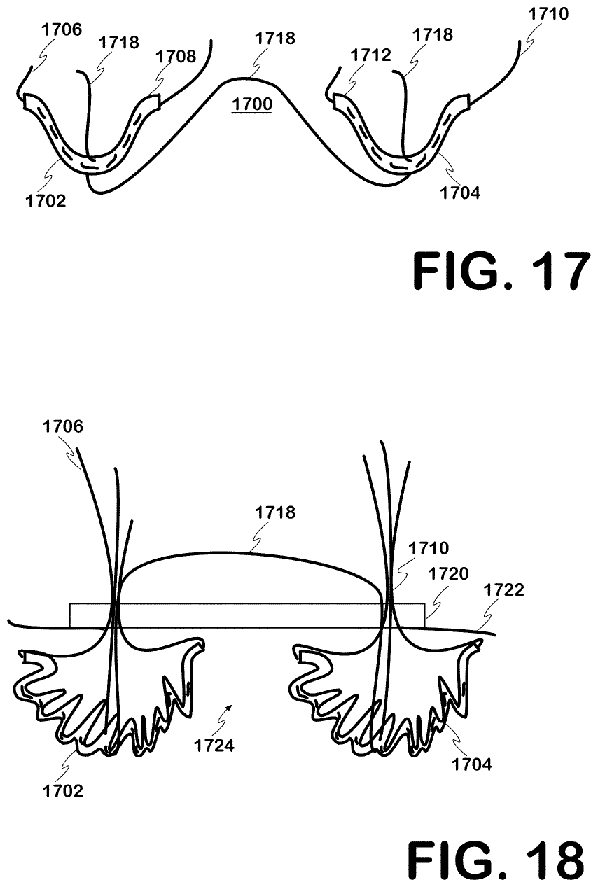

FIG. 17 illustrates another embodiment of a suture staple using multiple anchors as depicted in FIG. 15. The illustrated surgical device is a novel technique that transforms prior art anchors into a suture staple that can be used in tissue repairs and other surgical procedures. The suture staple 1700 includes two anchors 1702 and 1704. Each anchor includes two elements, a material that is to be cinched and a stitch that is used to cinch the cinchable material. In the embodiment presented in this FIG. 17, as well as the related embodiments described herein, the material utilized for the cinchable material and the cinching stitch can be constructed from a soft material, such as a thread, nylon or other suitable material such as is common in the art with sutures (i.e., most modern sutures are synthetic, including the absorbable polyglycolic acid, polylactic acid, Monocryl and polydioxanone as well as the non-absorbable nylon, polyester, PVDF and polypropylene as non-limiting examples). In the embodiments illustrated herein, the cinchable material is in the form of a tube with the cinch stitch passing through the center of the tube or being stitched along the sides of the tube. However, in some embodiments, the cinchable material maybe solid and the cinching stitch may be passed through the cinchable material, tacked at locations along the outer surface of the cinchable material, pass through loops attached to the outer surface of the cinchable material, and/or weaved through the cinchable material as a few non-limiting examples. In some embodiments, the cinchable material may be woven or non-woven fabric, braded suture strands or any of a variety of materials that easily chinches. In general, the anchors are simply constructed such that once inserted within a bone cavity, applying pressure to one or more of the cinching suture strands causes the cinching material to gather up into a ball or wad that is too large to fit back through the aperture in the cortex for a given amount of pressure.

In the illustrated embodiment, the cinching material is a hollow tube 1708 constructed of any of the variety of afore-mentioned materials or equivalents thereof. A cinch stitch 1706 passes through a hollow tube 1708 of anchor 1702 and a cinch stitch 1710 passes through a hollow tube 1712 of anchor 1702. The suture staple then includes a mattress stitch 1718 that can either be attached to the hollow tubes 1708 and 1712 or that can pass through an aperture in the tubes 1708 and 1712 and extend upwardly along with the ends of the cinch stitches 1706 and 1710. Thus, the mattress 1718 runs between the two anchors 1702 and 1704 creating a two-point suture staple. It should be appreciated that more than two anchors could be used in a similar configuration. Further, it should also be appreciated that an array of anchors and mattress stitches could be used to create a mesh-like device.

FIG. 18 illustrates the suture staple of FIG. 17 in a deployed state. In operation, each of the anchors 1702 and 1704 are driven through a membrane, such as the tissue 1720 and through the cortex 1722 of a bone and into the internal cavity 1724. The cinch stitch 1706 can then be pulled to cause the tube 1702 to ball up within the cavity 1724. The ends of the cinch stitch 1706 can then be tied to secure the anchor in place. Similarly, the ends of the cinch stitch 1710 can be pulled to cause the tube 1704 to ball up within the internal cavity 1724. The ends of the cinch stitch 1710 can then be tied to secure the anchor in place. At this point, the mattress stitch 1718 thus extends from the anchor 1702, up through the cortex 1722 and the tissue 1720, then back down through the tissue 1720 and cortex 1722 to the anchor 1704. In embodiments that have the mattress stitch 1718 secured to the tube 1708 and 1712, the anchors 1702 and 1704 are positioned such that the mattress stitch is held snugly against the membrane 1720 thus holding it to the cortex 1722 surface. In embodiments in which the mattress stitch 1718 passes through an aperture of the tubes 1708 and 1712, the mattress stitch 1718 can be pulled and tied to the cinch stitches 1706 and 1710 to tighten down on the membrane 1720. Thus, in this later embodiment, the free stitch running between the two prongs or anchors allow for further tensioning of the repair. Thus, rather than a suture staple with two prongs having a fixed length suture between them, this embodiment allows the length of the suture to be adjusted to any length. Adding the ability to have a free stitch running between the two prongs/anchor points improves on the utility of the device and can eliminate the requirement of having differently sized suture staples. In some embodiments, the anchors can be constructed out of suture material. The suture material acts as an anchor when driven into the bone as they will “ball up” larger than the initial hole through which they are inserted and not be able to be pulled out. There are numerous “all-suture anchors” currently on the market (such as the Con-Med Y-Knot), however, the present embodiments utilize these suture anchors to create a suture staple. The cinch stitch 1706/1710 may run freely through the interior of the anchor tubes 1702/1704, may be attached at positions along the interior of the anchor tubes 1702/1704, may be threaded in and out through the surface of the anchor tubes 1702/1704 or may include a knot or stop on either end of the cinch stitch, as a few non-limiting examples.

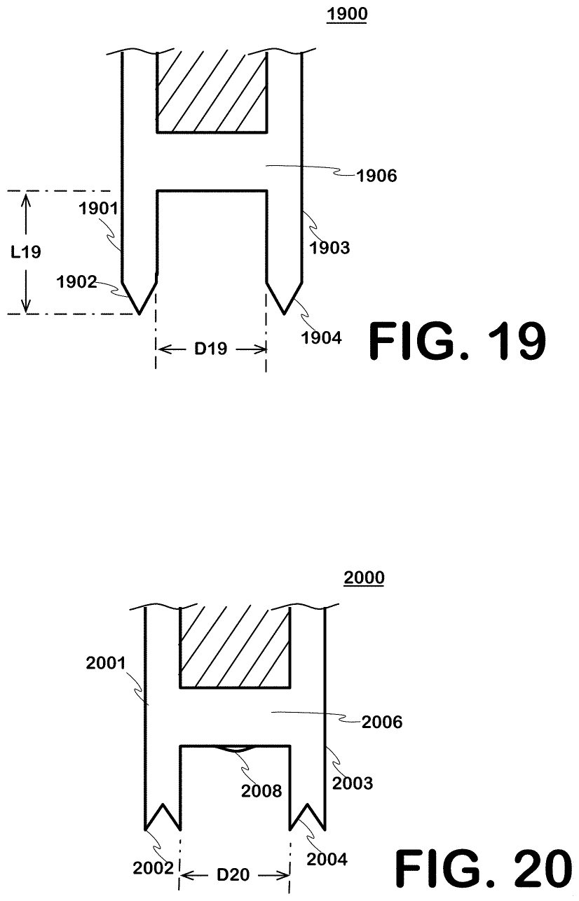

FIG. 19 illustrates an awl or punch to be used to prepare the bone for the implanting of a suture staple in the configuration illustrated in FIG. 17. The awl is used to create penetrations through the cortex and into the internal cavity of the bone. The awl 1900 is illustrated as including a first leg 1901 and a second leg 1903. The first leg 1901 and the second leg 1903 terminate on one end with a sharp point or edge, respectively first point 1902 and second point 1904. The first leg 1901 and the second leg 1903 are separated by an adjoining support structure 1906. The adjoining support structure holds the first leg 1901 and the second leg 1903 at a fixed distance D from each other. In operation, the awl 1900 is used in preparations for implanting of a two-anchor staple suture, such as the embodiment illustrated in FIG. 17. The first point 1902 and the second point 1904 can be positioned at the desired location for the suture and then driven through the cortex and into the internal cavity of the bone by striking the top of the awl 1900 with a hammer or blunt force instrument. It will be appreciated that the awl could also be included in a staple gun style configuration in which the anvil is actuated by a trigger or a CO2 cartridge, etc.

The adjoining support structure 1906 operates as a stop for the penetration of the awl 1900. For instance, once the adjoining structure 1906 comes in contact with the membrane and/or cortex, the awl does not travel further into the bone. In the illustrated embodiment, the travel distance of the first point 1902 and the second point 1904 is L19.

It should be appreciated that the awl 1900 can be configured with any distance D19, and such it is anticipated that depending on the application, implant tools with various values of D19 may be utilized for implanting a staple suture. Further, in some embodiments the implant tool can be adjusted over a range of distances D19 using a variety of techniques, such as a telescoping adjoining support structure or a multi-piece sliding adjoining support structure. In such embodiments, detents and protrusions on the sliding pieces of the adjoining support structure could be used to hold the anchor attachments at particular discrete distances D19 or a screw or clamp can be used to secure the sliding elements at any of a plurality of positions or two separate pieces may be threaded such that they can be screwed together like a nut and bolt.

It should also be appreciated that awls can be constructed for varying values of L19. Further, in some embodiments the awl may be adjustable such that the value of L19 can be set to different values depending on the particular application. One such technique is for the legs to include a screw mechanism to lengthen or shorten the value of L19. In other embodiments, the supporting structure 1906 may be moveable or adjustable up and down the lengths of the first leg 1901 and the second leg 1903, such as using pins that go through the legs and into the ends of the support structure or having threaded legs with threaded receptacles in the supporting structure 1906, as non-limiting examples. Further, it should be appreciated that in some applications, the length of the first leg 1901 and the second leg 1903 may need to be different values. The sliding or adjustable support structure could be used to accommodate such requirements.

FIG. 20 illustrates an implant tool for deploying suture staples, such as the suture staple illustrated in FIG. 17. The implant tool 2000 is illustrated as including a first anchor attachment 2002 and a second anchor attachment 2004. The first anchor attachment 2002, situated on the end of a first implant tool leg 2001 and the second anchor attachment 2004, situated on the end of a second implant tool leg 2003, are separated by an implant tool adjoining support structure 2006. The adjoining support structure holds the first anchor attachment 2002 and the second anchor attachment 2004 a fixed distance D20 from each other. The adjoining support structure 2006 further includes a horizontal mattress clip 2008 that is used to secure or hold the horizontal mattress in place during the implant process. In operation, a two-anchor staple suture, such as the embodiment illustrated in FIG. 17, can be loaded into the implant tool 2000 by placing a first anchor 1702 into the first anchor attachment 2002 and a second anchor attachment 1704 into the second anchor attachment 2004. The horizontal mattress stitch 1718 can then be attached to the horizontal mattress clip 2008. It should be appreciated that the implant tool 2000 can be configured with any distance D20, and as such it is anticipated that depending on the application, implant tools with various values of D20 may be utilized for implanting a staple suture. Further, in some embodiments the implant tool can be adjusted over a range of distances D20 using a variety of techniques, such as a telescoping adjoining support structure, threaded mating structures or a multi-piece sliding adjoining support structure. In such embodiments, detents and protrusions on the sliding pieces of the adjoining support structure could be used to hold the anchor attachments at particular discrete distances D20 or a screw or clamp can be used to secure the sliding elements at any of a plurality of positions.

In operation, the values of D19 and D20 are substantially the same. The awl 1900 is used to create penetrations into the bone and then the loaded implant tool 2000 is used to place the anchors into the penetrations. At this point the cinch stitches can be pulled to secure the anchors into position and leaving the mattress suture holding tissue to the bone between the first and second legs of the implant tool 2000.

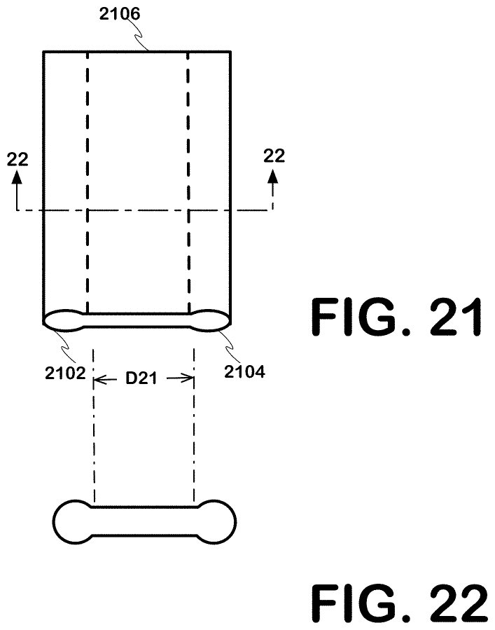

FIG. 21 illustrates a cannula that can be used in conjunction with the implant tool of FIG. 17 and the awl of FIG. 19. FIG. 22 illustrates a cross-sectional view of the cannula of FIG. 21 taken at line 22-22. The cannula includes a first pillar 2102 and a second pillar 2104. An adjoining shaft 2106 is used to hold the first pillar 2102 and the second pillar 2104 at a fixed distance D21 from each other. In operation, the cannula 2100 can be positioned over the tissue and bone that is to be stapled with the suture staple. Once in position, the awl 1900 can be inserted into the cannula 2100 and then struck with a blunt tool to drive the points 1902 and 1904 through the tissue and cortex and into the internal cavity of the bone. Next, the awl 1900 can be removed from the cannula 2100 and the implant tool 2000 can be loaded with the two-anchor suture staple and then slid into the pillars of the cannula 2100. Again, a blunt instrument may be used to force the ends of the implant tool 2000 into the bone penetrations. The cannula 2100 and the implant tool 2000 can then be extracted and the cinch stitches can be actuated to secure the anchors into position and leaving the mattress suture extending from one anchor to the next and over the tissue that is securely held to the bone.

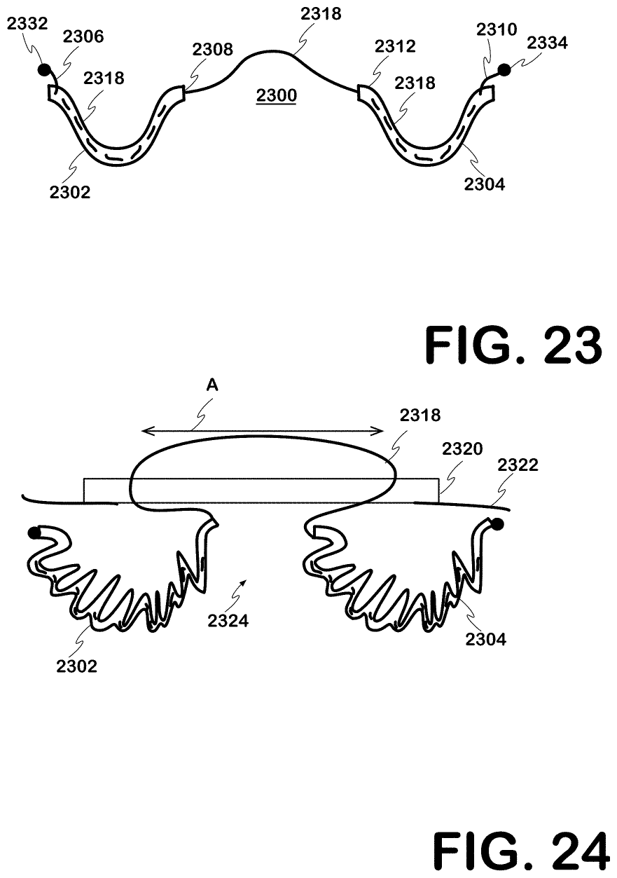

FIG. 23 illustrates another embodiment of the two-anchor suture staple. As illustrated in FIG. 23, the suture staple 2300 includes two anchors 2302 and 2304. In this embodiment, rather than cinch stitches, the mattress stitch 2318 runs through the anchor tubes 2308 and 2312. On each end of the mattress stitch 2318 is a bulge, knot, stop, etc. 2332 and 2334. FIG. 24 illustrates the suture staple embodiment of FIG. 23 in a deployed configuration. It can be seen by examining FIG. 23 and FIG. 24 that in the deployment of the suture staple 2300, the stops 2332 and 2334 operate to cinch the anchors 2302 and 2304 during implanting the suture staple as the mattress stitch 2318 tightens over the tissue 2320 the suture tubes 2308 and 2312 ball up within the internal cavity 2324 below the cortex 2322. It should also be appreciated that the mattress stitch 2318 can be cut proximate to area A and then used to further cinch the anchors 2302 and 2304 and then tied or otherwise connected to each other.

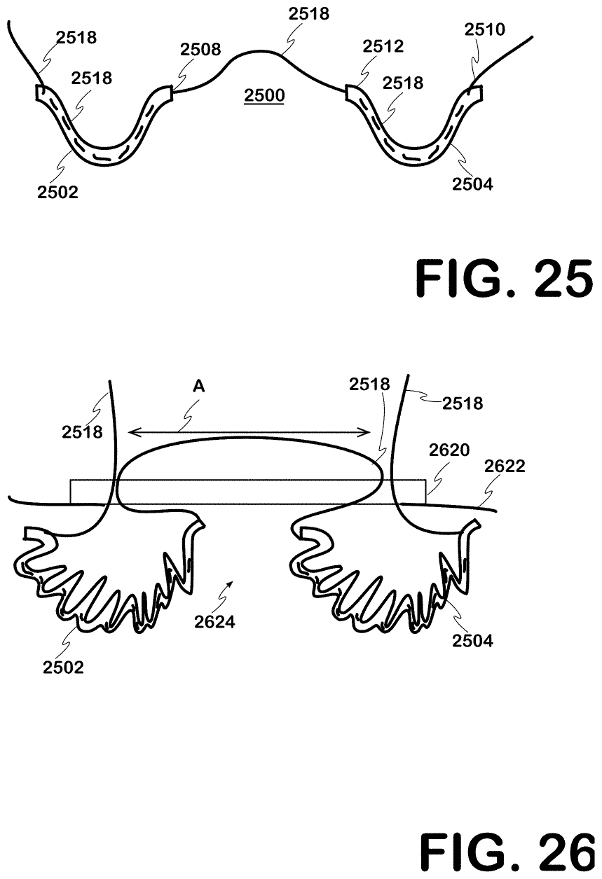

FIG. 25 is yet another embodiment of a two-anchor suture staple. As illustrated in FIG. 25, the suture staple 2500 includes two anchors 2502 and 2504. In this embodiment, rather than cinch stitches, the mattress stitch 2518 runs through the anchor tubes 2508 and 2512. FIG. 26 illustrates the suture staple of FIG. 25 in a deployed or implanted state. The free-standing ends of the mattress stitch 2518, and the central portion of the mattress stitch 2518 extend through the aperture passing through the tissue 2620, the cortex and into the internal cavity 2524 of the bone. Thus, it can be appreciated that the mattress stitch 2518 can be pulled from either end to tighten the mattress suture against the tissue to secure it to the cortex. The free ends of the mattress stitch 2518 can then be tied to each other, knotted, tied to a soft, pliable stop (such as a silicone ball), tied to the central portion of the mattress suture, etc.

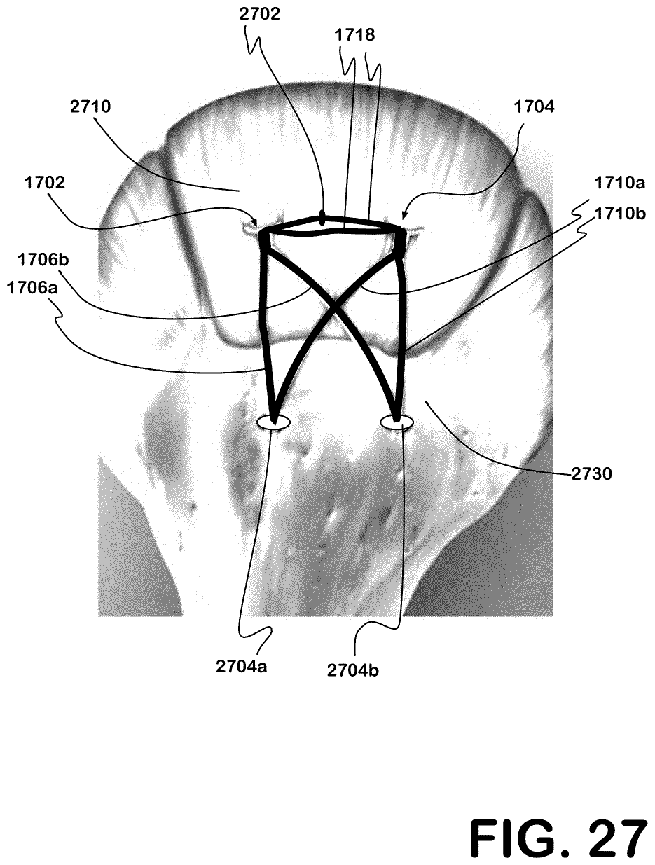

FIG. 27 illustrates the application of the embodiment of the suture staple of FIGS. 17-18 in use for a rotator cuff repair. In the illustrated application, anchor 1702 and anchor 1704 have been implanted through the supraspinatus muscle 2710 and into the humerus bone 2730 (not seen as it is below the surface of the supraspinatus muscle 2710). The cinch stitch 1706 passes through the hollow tube 1708 of anchor 1702 and the cinch stitch 1710 passes through a hollow tube 1712 of anchor 1702. Once the anchors 1702 and 1704 are implanted within the internal cavity of the humerus bone 2370, the cinch stitches 1706 and 1710 can be pulled with one leg of stitch 1706 (1706a) being tied or secured to another anchor 2704a, and the other leg of cinch stitch 1706 (1706b) being tied or secured to another anchor 2704b. Similarly, one leg of cinch stitch 1710 (1710a) can be secured to anchor 2704a and the other leg, 1710b secured to anchor 2704b. The mattress stitch 1718 then is pulled secure or tight and then tied off at knot 2702 to provide the horizontal mattress at 1718. For embodiments in which the mattress stitch 1718 is actually secured directly to the tubes of the anchors 1702 and 1704, then only a single mattress stitch runs between the anchors 1702 and 1704, however, other embodiments may utilize additional mattress stitches to provide additional strength and reliability.

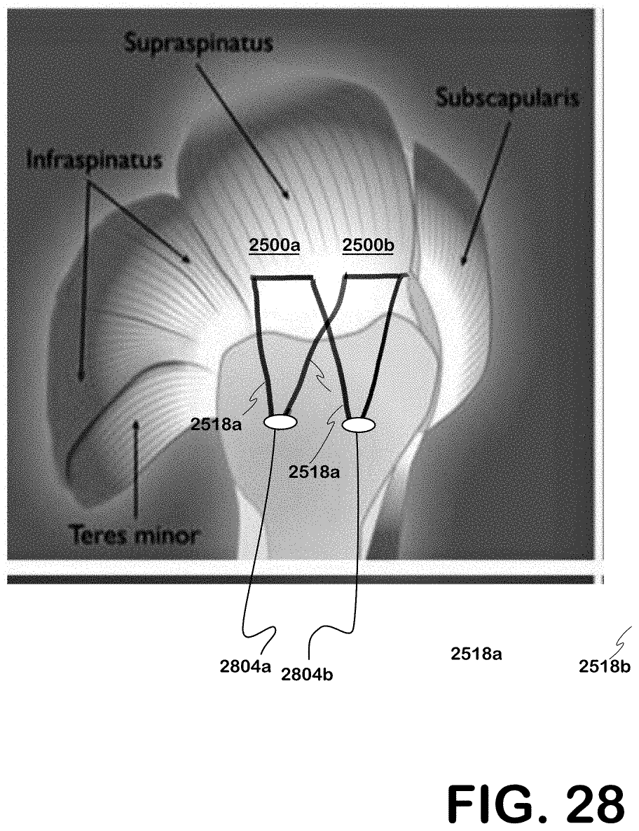

FIG. 28 illustrates the application of the embodiment of the suture staple of FIGS. 25-26 in use for a double rotator cuff repair. In embodiments, such as the one depicted in FIG. 25 and FIG. 26, the cinch stitch and the mattress stitch are all one piece and thus, one end of the mattress stitch 2518 would be secured to an anchor and the other end of the mattress stitch 2518 would also be secured to an anchor. In the illustrated embodiment, two suture staples 2500 are utilized (illustrated as 2500a and 2500b). The mattress stitch 2518a is thus stretched across the tissue between the anchors of the staple 2500a, and then the ends are secured to other anchors (i.e., anchors 2804a and/or 2804b) or the two ends can simply be knotted or tied together, secured to the same anchor or otherwise secured together to create a double mattress. Similarly, the suture anchor 2500b can be implanted and the mattress stitch 2518b can stretch across the tissue between the anchors of the staple 2500b and the ends can be secured to other anchors (i.e., anchors 2804a and/or 2804b) or the two ends can simply be knotted or tied together, secured to the same anchor or otherwise secured together to create a double mattress.

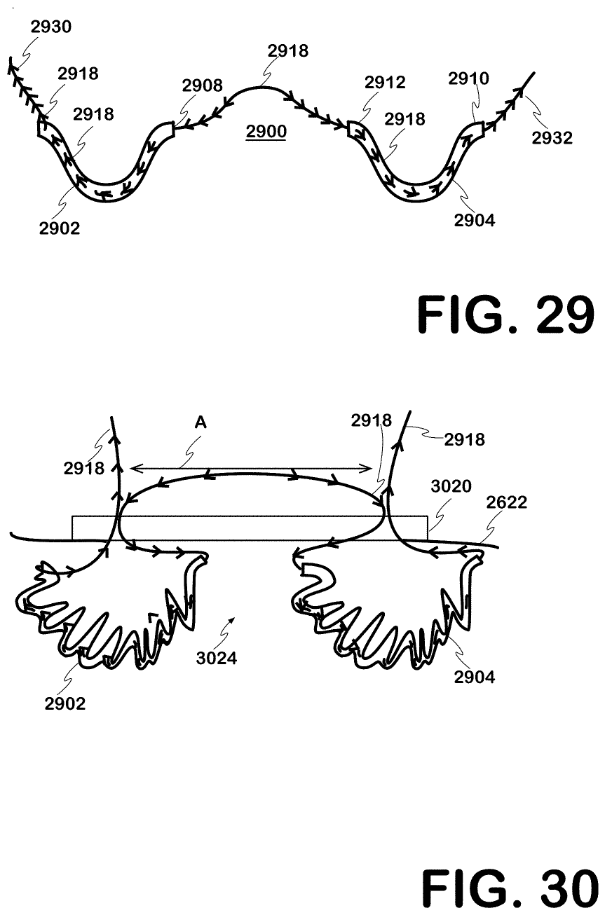

FIG. 29 is yet another embodiment of a two-anchor suture staple. As illustrated in FIG. 29, the suture staple 2900 includes two anchors 2902 and 2904. In this embodiment, rather than cinch stitches, the mattress stitch 2918 runs through the anchor tubes or segments of cinching material 2908 and 2912. The mattress stitch 2918 is illustrated as a barbed suture. The barbed suture is illustrated as including a plurality of outward facing barbs 2930 and 2932. The barbs 2930 running from approximately the center of the mattress stitch 2918 through tube 2902 are oriented in a first direction, while the barbs 2932 running from approximately the center of the mattress stitch 2918 though the tube 2904 face a second direction that is opposite to the first direction. Thus, the barbs are facing outwards from the center of the mattress stitch, wherein outwards facing means that the point where the barb meets the surface of the mattress stitch or suture is closest to the end of the mattress stitch or suture, and the barb extends away from the surface of the mattress stitch or suture towards the middle section of the mattress stitch or suture. Thus, in the illustrated embodiment, the barbed suture is bi-directional. In some embodiments, a uni-direction barb stitch may be utilized as well. The barbs on each end of the mattress stitch 2918 are oriented such that the mattress stitch 2918 can be pulled through the tubes or segments of cinching material 2904 and 2904 in opposing directions to tighten the mattress stitch 2918 over the tissue but, the barbs prevent or retard the ability for the mattress stitch 2918 to be pulled through the tube or cinching material in the opposite direction (which would have the effect of loosening the mattress stitch.

The barbed sutures used in the embodiment illustrated in FIG. 29 may also be used in other embodiments such as suture staples that include one or more suture staples. The barbed sutures can be fabricated from a variety of materials including non-absorbable plastics and absorbable plastics, polyethylene and fiber wire as a few non-limiting examples. In some embodiments, the tube can be constructed of a braded material or other material that is more conducive to being snagged by the barbs. Preferably, the barbs 2930 and 2932 in conjunction with the tubes 2902 and 2904 provide a holding strength of 50 pounds of pressure, or 45 to 55 pounds of pressure, or 40 to 60 pounds of pressure, or greater than at least 50 pounds of pressure. In some embodiments, suture staples with less holding strength can be used and multiple suture staples can be used to help distribute the force that is applied to the repaired tissue. The barbs, while illustrated as being on opposing sides of the mattress stitch, may actually exist on one side of the mattress stitch, multiple sides of the mattress stitch, be aligned, or staggered, exist in a spiraled configuration as a few non-limiting examples. Specific examples of barbed stitches include the STRATAFIX products sold by JOHNSON & JOHNSON and THE QUILL products sold by SURGICAL SPECIALTIES.

FIG. 30 illustrates the suture staple of FIG. 29 in a deployed or implanted state. The free-standing ends of the mattress stitch 2918, and the central portion of the mattress stitch 2918 extend through the aperture passing through the tissue 3020, the cortex and into the internal cavity 3024 of the bone. Thus, it can be appreciated that the mattress stitch 2918 can be pulled from either end to tighten the mattress suture against the tissue to secure it to the cortex as the segments of cinching material are bunched up to create a ball or knot that is too large to be pulled through the cortex. The free ends of the mattress stitch 2918 can then be tied to each other, knotted, tied to a soft, pliable stop (such as a silicone ball), tied to the central portion of the mattress suture, tied to another anchor, tied to ends of stitches from neighboring staples, etc.

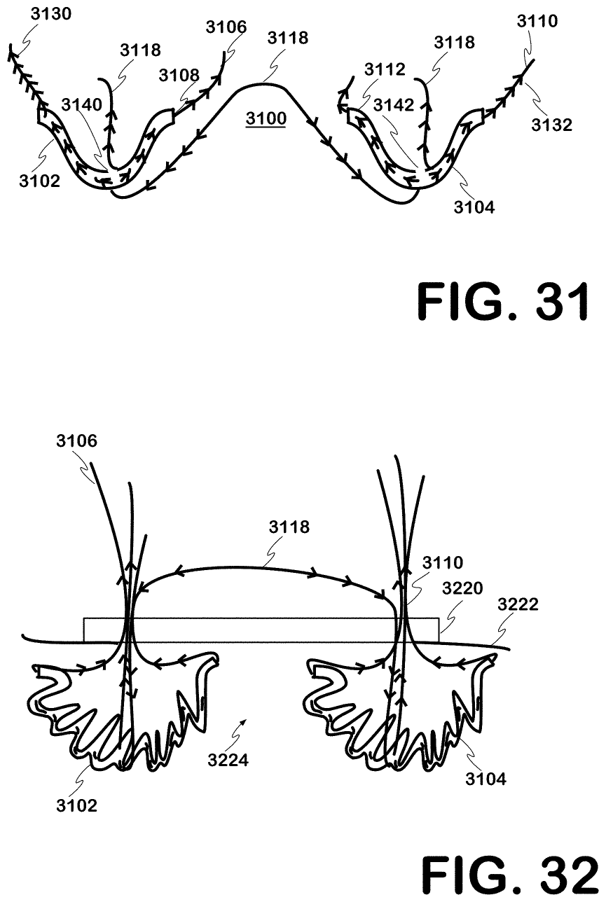

FIG. 31 is yet another embodiment of a two-anchor suture staple. As illustrated in FIG. 31, the suture staple 3100 includes two anchors 3102 and 3104. The suture staple 3100 includes two anchors 3102 and 3104. Each anchor includes two elements, a material that is to be cinched and a stitch that is used to cinch the cinchable material. The material utilized for the cinchable material or segment and the cinching stitch can be constructed from a soft material, such as a thread, nylon or other suitable material such as is common in the art with sutures (i.e., most modern sutures are synthetic, including the absorbable polyglycolic acid, polylactic acid, Monocryl and polydioxanone as well as the non-absorbable nylon, polyester, PVDF and polypropylene as non-limiting examples). In the illustrated embodiment, the cinching material is a hollow tube constructed of any of the variety of afore-mentioned materials or equivalents thereof. A cinch stitch 3106 passes through cinch material or segment in the form of a hollow tube 3108 of anchor 3102 and a cinch stitch 3110 passes through cinch material or segment in the form of a hollow tube 3112 of anchor 1704. The suture staple 3100 then includes a mattress stitch 3118 that can either be attached to the hollow tubes 3108 and 3112 or that can pass through an aperture in the tubes 3108 and 3112 and extend upwardly along with the ends of the cinch stitches 3106 and 3110. Thus, the mattress 3118 runs between the two anchors 3102 and 3104 thus creating a two-point suture staple. It should be appreciated that more than two anchors could be used in a similar configuration. Further, it should also be appreciated that an array of anchors and mattress stitches could be used to create a mesh-like device.

The cinch stitch 3106 is illustrated as including barbs 2930 on the outer surface of the cinch stitch 3106. In the illustrated embodiment, the barbs are outward facing relative to a mid-point of the cinch stitch 3106. As such, the cinch stitch 3106 can be pulled through the cinching material 3108 to cause the cinching material to cinch or ball up but, the barbs 2930 prevent or retard the ability of the cinch stitch 3106 to be pulled in the opposite direction. Cinch stitch 3110 is similarly structured with barbs 3132. To facilitate threading the cinch stitches 3106 and 3110 through the cinching material 3108 and 3112 respectively, an aperture 3140 and 3142 in the cinching material 3108 and 3112 respectively may be utilized for inserting the cinch stitches.

FIG. 32 illustrates the suture staple of FIG. 31 in a deployed state. In operation, each of the anchors 3102 and 3104 are driven through a membrane, such as the tissue 3220 and through the cortex 3222 of a bone and into the internal cavity 3224. The cinch stitch 3106 can then be pulled to cause the cinching material or segment 3102 to ball up within the cavity 3224. The ends of the cinch stitch 3106 can then be tied to secure the anchor in place. Similarly, the ends of the cinch stitch 3110 can be pulled to cause the cinching material or segment 3104 to ball up within the internal cavity 3224. The ends of the cinch stitch 3110 can then be tied to secure the anchor in place. At this point, the mattress stitch 3118 thus extends from the anchor 3102, up through the cortex 3222 and the tissue 3220, then back down through the tissue 3220 and cortex 3222 to the anchor 3104. In embodiments that have the mattress stitch 3118 secured to the cinching material or segment 3108 and 3112, the anchors 3102 and 3104 are positioned such that the mattress stitch is held snugly against the membrane 3220 thus holding it to the cortex 3222 surface. In embodiments in which the mattress stitch 3118 passes through an aperture of the tubes 3108 and 3112, the mattress stitch 3118 can be pulled and tied to the cinch stitches 3106 and 3110 (or each other) to tighten down on the membrane 3220. Thus, in this later embodiment, the free stitch running between the anchors allows for further tensioning of the repair. Thus, rather than a suture staple with two prongs having a fixed length suture between them, this embodiment allows the length of the suture to be adjusted to any length. Adding the ability to have a free stitch running between the two anchor points improves on the utility of the device and can eliminate the requirement of having differently sized suture staples. In some embodiments, the anchors can be constructed out of suture material. The suture material acts as an anchor when driven into the bone as they will “ball up” larger than the initial hole through which they are inserted and not be able to be pulled out. There are numerous “all-suture anchors” currently on the market (such as the Con-Med Y-Knot), however, the present embodiments utilize these suture anchors to create a suture staple. The cinch stitch 3106/3110 may run freely through the interior of the cinching material or anchor tubes 3102/3104, may be attached at positions along the interior of the anchor tubes 3102/3104, may be threaded in and out through the surface of the anchor tubes 3102/3104, may include a knot or stop on either end of the cinch stitch, as a few non-limiting examples.

Thus, in any of these applications it will be appreciated that a horizontal mattress stitch is secured to two anchors implanted in the interior cavity of the bone (such as a humerus bone) and the horizontal mattress stitch extends between the two anchors thus securing the muscle or tissue (i.e., supraspinatus muscle) to the bone.

It should be appreciated that in each of the embodiments illustrated in FIGS. 17, 18, 23, 24, 25, 26, 29 and 31, while only two anchors are illustrated, any number of anchors could actually be used in various embodiments of the present invention. For instance, three anchors could be used, as well as an array of N-M anchors to create a mesh of mattress sutures.

It should be appreciated that while the awl 1900, implant tool 2000 and cannula 2100 have been described as supporting a two-anchor staple, the same principles can be applied to create an N anchor awl, implant tool and cannula.

It should be appreciated that while a tissue repair staple has been described as including one, two or three prongs, any number of prongs could also be used in the tissue repair or in other surgical procedures. It should also be appreciated that in some embodiments, rather than driving the prongs into the bone, a hole can be drilled and the prongs can be inserted into the bones and held in place by friction or an adhesive. It should also be appreciated that in some embodiments, the prongs may be threaded and thus screwed into the bone or into a drilled hole within the bone.

Further, it should be appreciated that rather than using a blunt force instrument to drive the staple into the bone, a staple gun could be constructed to hold the staple in position and then drive the stable prongs into the tibia by pulling the trigger.

The various embodiments presented herein have focused on the use of the disclosed embodiments within the context of rotator cuff and meniscus root repairs. However, it should be appreciated that the features, elements and aspects of the various described embodiments may also be applied to other soft tissue repairs or in any situation in which there needs to be a rigid fixation of soft tissue to bone. As non-limiting examples, different sizes and shapes of the suture staple can be created for purposes of rotator cuff repair, ACL reconstruction and primary ligament repair. The various embodiments differ from the prior art, such as metal anchors, in that the suture staple can serve as a horizontal mattress suture when passed directly into and through a soft tissue (eg. ligament/tendon/muscle) as opposed to just stapling around it like the metal staples. Advantageously, the various embodiments thus provide the overall construct additional strength as the tendon/ligament/muscle could not slip out from under the staple.

Further, while the rigid portion of the various embodiments of the suture staple has been described as being constructed from stainless steel, composite material or cobalt steel, it should be appreciated that other rigid materials may also be utilized in the various embodiments presented and equivalents thereof. For instance, the rigid portion of the suture staples could be constructed with a rigid plastic, such as “PEEK”. PEEK, which stands for (polyetheretherketone) is a high-performance engineering plastic with outstanding resistance to harsh chemicals and excellent mechanical strength and dimensional stability. It offers hydrolysis resistance to steam, water and sea water. PEEK has the ability to maintain stiffness at high temperatures and is suitable for continuous use at temperatures up to 338 degrees F. (170 degrees C.). This engineering plastic has a proven track record in challenging environments such as aerospace, oil and gas and semiconductors and is also useful in surgical implants. Further, some embodiments may utilize a bioabsorbable substance such as poly-L-lactide (PLLA), biphasic calcium phosphate (BCP) and/or hydroxyapatite, as well as variants and hybrids of these materials as well as other materials.

It should also be appreciated that the size, shape, length, thickness, etc. of the various elements of the described embodiments may be modified to make embodiments more suitable for other applications without departing from the spirit and scope of the present invention.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

It will be appreciated by persons skilled in the art that the present invention is not limited by what has been particularly shown and described herein above. Rather the scope of the invention is defined by the claims that follow.

What is claimed is:

1. An apparatus for repairing tissue comprising:
a first segment of cinching material having a first end and a second end;
a second segment of cinching material having a first end and a second end;
a mattress stitch wherein one end of the mattress stitch is associated with the first segment and a second end of the mattress stitch is associated with the second segment;
a cinching stitch that is associated with at least the first segment or the second segment such that pressure applied to the cinching stitch causes at least the first segment or the second segment to be cinched; and
a plurality of outward facing barbs on an outer surface of the cinching stitch, wherein the cinching stitch can be pulled through the at least first segment or second segment in a first direction but the plurality of outward facing barbs retard the ability for the cinching stitch to be pulled through the at least first segment or second segment in a second direction.

2. The apparatus of claim 1, wherein the mattress stitch and the cinching stitch are the same and the cinching stitch is associated with the first segment and the second segment such that the cinching stitch passes through the first end of the first segment, the second end of the first segment, the first end of the second segment and the second end of the second segment such that a first end of the cinching stitch extends from the first end of the first segment and a second end of the cinching stitch extends from the second end of the second segment and a middle section of the cinching stitch extends from the second end of the first segment to the first end of the second segment; and
the plurality of outward facing barbs on the outer surface of the cinching stitch extend from the middle section of the cinching stitch towards the first end of the cinching stitch and the second end of the cinching stitch.

3. The apparatus of claim 1, wherein the cinching stitch includes two cinching stitches with a first cinching stitch associated with the first segment and the second cinching stitch associated with the second segment, and wherein the plurality of outward facing barbs face outward from a central area of each of the first and second cinching stitches, wherein the first cinching stitch passes through the first end of the first segment and the second end of the first segment and the second cinching stitch passes through the first end of the second segment and the second end of the second segment.

4. The apparatus of claim 3, wherein the mattress stitch extends through a cross section of the first segment between the first end and the second end of the first segment and a cross section of the second segment between the first end and the second end of the second segment.

5. The apparatus of claim 4, wherein the mattress stitch includes a plurality of outward facing mattress barbs relative to a middle area of the mattress stitch.

6. A method for performing tissue repair to secure a tissue to a bone, the method comprising the actions of:
positioning an apparatus over a section of tissue and driving the apparatus through or around the section of tissue and through a cortex of a bone proximate to the section of tissue, wherein the apparatus comprises:
a first segment of cinching material having a first end and a second end;
a second segment of cinching material having a first end and a second end;
a mattress stitch wherein one end of the mattress stitch is associated with the first segment and a second end of the mattress stitch is associated with the second segment;
a cinching stitch that is associated with at least the first segment or the second segment such that pressure applied to the cinching stitch causes at least the first segment or the second segment to be cinched; and
a plurality of outward facing barbs on the surface of the cinching stitch, wherein the cinching stitch can be pulled through the at least first segment or second segment in a first direction but the plurality of outward facing barbs retard the ability for the cinching stitch to be pulled through the at least first segment or second segment in a second direction; and
applying pressure to the cinching stitch to cause the at least first segment or the second segment to cinch into a ball, whereby the at least first segment or second segment is secured below the cortex of the bone.

7. The method of claim 6, wherein the mattress stitch and the cinching stitch are the same and the cinching stitch is associated with the first segment and the second segment such that the cinching stitch passes through the first end of the first segment, the second end of the first segment, the first end of the second segment and the second end of the second segment such that a first end of the cinching stitch extends from the first end of the first segment and a second end of the cinching stitch extends from the second end of the second segment and a middle section of the cinching stitch extends from the second end of the first segment to the first end of the second segment; and
the plurality of outward facing barbs on the outer surface of the cinching stitch extend from the middle section of the cinching stitch towards the first end of the cinching stitch and the second end of the cinching stitch; and
the action of applying pressure to the cinching stitch further comprises pulling the first end of the cinching stitch a first direction and the second end of the cinching stitch a second direction substantially opposite to the first direction, whereby the first segment and the second segment are cinched below the cortex of the bone and the cinching stitch passes over the section of tissue thereby securing the section of tissue to the bone.

8. The method of claim 6, wherein the cinching stitch includes two cinching stitches; and
wherein a first cinching stitch is associated with the first segment and a second cinching stitch is associated with the second segment; and
wherein the plurality of outward facing barbs face outward from a central area of each of the first and second cinching stitches; and
wherein the first cinching stitch passes through the first end of the first segment and the second end of the first segment such that a first end of the first cinching stitch extends from the first end of the first segment and a second end of the first cinching stitch extends from the second end of the first segment; and
wherein the second cinching stitch passes through the first end of the second segment and the second end of the second segment such that a first end of the second cinching stitch extends from the first end of the second segment and a second end of the second cinching stitch extends from the second end of the second segment; and
the action of applying pressure to the cinching stitch further comprises pulling the first end and the second end of the first cinching stitch in opposing directions and pulling the first end and the second end of the second cinching stitch in opposing directions.

9. The method of claim 8, further comprising the action of securing the first end and second end of the first cinching stitch and the first end and the second end of the second cinching stitch to one of one or more anchors secured to the bone proximate to the apparatus.

10. The method of claim 8, wherein the mattress stitch extends through a cross section of the first segment between the first end and the second end of the first segment and a cross section of the second segment between the first end and the second end of the second segment such that the first end of the mattress stitch extends through a mid-point of the first segment and the second end of the mattress stitch extends through a mid-point of the second segment; and
further comprising the action of pulling the first end and the second end of the mattress stitch in opposing directions.

11. The method of claim 10, wherein the mattress stitch includes a plurality of outward facing mattress barbs relative to a middle area of the mattress stitch and further comprising the action of securing the first end of the mattress stitch to the second end of the mattress stitch.

12. The method of claim 10, wherein the mattress stitch includes a plurality of outward facing mattress barbs relative to a middle area of the mattress stitch and further comprising the action of securing the first end of the mattress stitch to a first anchor in the bone proximate to the apparatus and securing the second end of the mattress stitch to a second anchor in the bone proximate to the apparatus.

13. The method of claim 10, further comprising the action of securing the first end of the mattress stitch to the second end of the mattress stitch.

14. The method of claim 10, further comprising the action of securing the first end of the mattress stitch to a first anchor in the bone proximate to the apparatus and securing the second end of the mattress stitch to a second anchor in the bone proximate to the apparatus.
